(19) United States
(12) Patent Application Publication
(10) Pub. No.: US 2007/0099992 A1
(43) Pub. Date: May 3, 2007

(54) Direct application of vitamin C to the lung tissue and respiratory passages by use of an asthma medication-type inhaler or similar inhaler

(76) Inventor: Lynn Carol Nocella Kane, Panama City (PA)

Correspondence Address: Lynn Carol Nocella Kane, Apartado 0832-1648, World Trade Center, Panama City, PA

(21) Appl. No.: 11/265,748

(22) Filed: Nov. 3, 2005

(51) Int. Cl. A61K 31/34; A01N 43/08

(52) U.S. Cl. 514/474

(57) ABSTRACT

An asthma medication—type inhaler or any other type inhaler filled with an aqueous or any other type solution of sodium ascorbate or any other form of vitamin C.

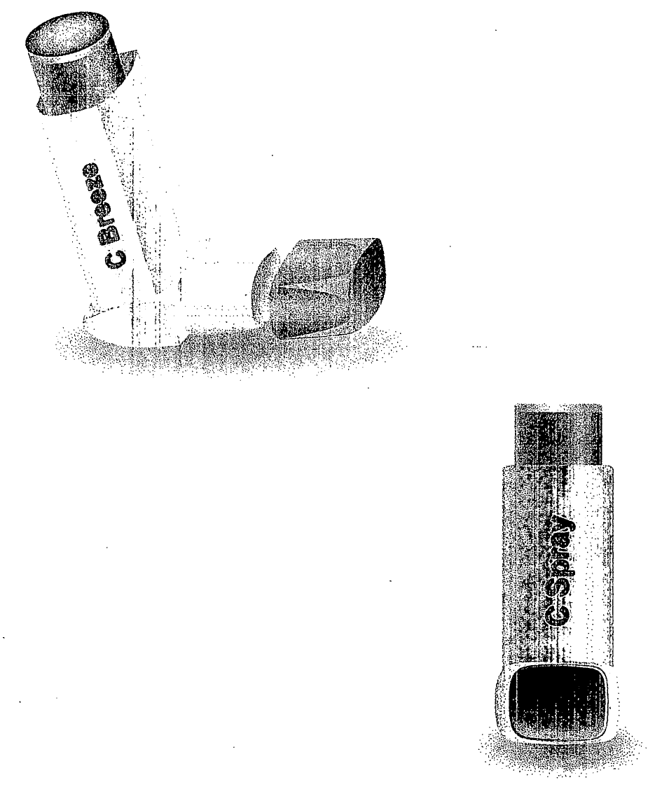

### SUMMARY

My invention is very simple. An asthma medication—type inhaler or similar inhaler will be filled with a solution of vitamin C, in the form of sodium ascorbate, to be applied in measured bursts of super-fine spray directly to the respiratory passages and lungs.

### DESCRIPTION

My invention is to make an aqueous solution of sodium ascorbate, which has a neutral pH and is the most soluble of all forms of vitamin C and apply it directly to the lungs and respiratory passages by way of an asthma medication—type inhaler or similar inhaler, thereby bipassing the digestive tract and administering a topical application of vitamin C directly to the tissue which most needs it, in the case of smoking damage or respiratory ailments. I would, however, want to patent the inhalable type of all forms of vitamin C, including but not limited to dehydroascorbic acid, D-ascorbic acid, L-ascorbic acid, ascorbic acid, 2,3-didehydro-L-threo-hexano-1,4-lactone, 3-oxo-L-gulofuranolactone, L-threo-hex-2-enonic acid gamma-lactone, L-3-keto-threo-hexuronic acid lactone, L-xylo-ascorbic acid, antiscorbutic vitamin, vitamin C with bioflavonoids, acerola vitamin C, rose hip vitamin C, all mineral ascorbates, and ascorbate and vitamin C metabolites.

The drawing page consists of a side view and frontal view of the inhalable vitamin C applicator.

I do not have the facilities to do the clinical studies and to package this product myself, and would need to work together with a company which manufactures vitamin products. But I do not want to approach anyone with this until I have the patent in hand.

1. What I claim as my invention is an asthma medication-type inhaler or any other type inhaler filled with a solution of any form of vitamin C, to be used to apply vitamin C directly to the lungs and respiratory passages.
